(19) United States
(12) Patent Application Publication
(10) Pub. No.: US 2013/0103006 A1
(43) Pub. Date: Apr. 25, 2013

(54) MESH PROTECTION SYSTEM

(71) Applicant: Brett Schleicher, New York, NY (US)

(72) Inventor: Brett Schleicher, New York, NY (US)

(73) Assignee: PALYON MEDICAL (BVI) LIMITED, Tortola (VG)

(21) Appl. No.: 13/276,469

(22) Filed: Oct. 19, 2011

(51) Int. Cl. A61M 5/142

(52) U.S. Cl. 604/891.1

(57) ABSTRACT

An improved safety feature for preventing overdosage to a patient is disclosed. In one embodiment, the feature includes an improved screen member that prevents the inadvertent entry of a needle having too large a diameter. The screen member includes a plurality of holes that are configured so as to rid unwanted dead spaces that can damage an injection device during injection. Another embodiment of the safety feature includes a plurality of pins situated so as to similarly create a plurality of openings for reception of certain sized injection devices. Methods of providing a bolus dose to a patient are also disclosed.

### BACKGROUND OF THE INVENTION

The present invention relates to implantable pumps, more particularly, an improved safety feature for preventing overdosage to a patient.

Implantable pumps have been well known and widely utilized for many years. Typically, pumps of this type are implanted into patients who require the delivery of medication or other fluids (hereinafter referred to as “active substances”) to specific areas of their body. For example, patients that are experiencing severe pain may require pain killers daily or multiple times per day. Absent the use of an implantable pump or the like, a patient of this type would be subjected to one or more painful injections of such active substances. In the case of pain associated with more remote areas of the body, such as spine, these injections may be extremely difficult to administer and particularly painful for the patient. Furthermore, attempting to treat conditions such as this through oral or intravascular administration of an active substance often requires higher doses and may cause severe side effects. Therefore, it is widely recognized that utilizing an implantable pump may be beneficial to both the patient and the treating physician.

Many implantable pump designs have been proposed. For example, U.S. Pat. No. 4,969,873 (“the '873 patent”), the disclosure of which is hereby incorporated by reference herein, teaches one such design. The '873 patent is an example of a constant flow pump, which typically includes a housing having two chambers, the first chamber for holding the active substance to be administered to the patient and the second chamber for holding a propellant. A flexible membrane separates the two chambers such that expansion of the propellant in the second chamber pushes the active substance out of the first chamber. This type of pump also typically includes an outlet opening connected to both the first chamber and a catheter or other delivery device for directing the active substance to the desired area of the body, a replenishment port for allowing refilling of the first chamber, and a bolus or catheter direct access port for allowing the direct introduction (i.e., a bolus dose) of an active substance through the catheter without having to pass through other portions of the pump. Bolus dosages can be important in treating moments of intolerable pain or other situations where a patient requires a quick does of an active substance. The replenishment and bolus ports are typically each covered by septa, which reseal after a filling device such as a needle, syringe, cannula or the like (hereinafter “an injection device”) is passed therethrough.

As pumps of this type are designed to provide a constant flow of active substance to a specific area of the body, they must be refilled periodically with the proper concentration of active substance suited for extended release. The overall volume of active substance to be contained in the first chamber is often significantly more than would be given to a patient in a single bolus dose. It has been a safety concern for some time that medication meant to refill the first chamber not be inadvertently administered as a bolus dose. In fact, the prior art is replete with different safety features for preventing such an inadvertent administration of an active substance.

For instance, U.S. Pat. No. 4,978,338 (“the '338 patent”), the disclosure of which is hereby incorporated by reference herein, teaches the use of multiple syringes each having an opening set forth at a different height. The openings in the syringes of the '338 patent are meant to be utilized in connection with a multiple septum pump design so that only a syringe having an opening at a certain height can be utilized to administer a bolus dose. U.S. Pat. Nos. 5,328,465 (“the '465 patent”) and 6,293,932 (“the '932 patent”), the disclosures of which are also hereby incorporated by reference herein, each teach pump structures overlying bolus ports that are sized to only allow injection devices of a certain diameter to be inserted therethrough. More particularly, the '465 patent teaches a screen member having a plurality of circular openings that will only allow injection devices of a size less than a predetermined diameter to pass therethrough, while the '932 patent teaches a conical depression having a single opening that is similarly sized to only allow injection devices having a certain diameter to pass therethrough. Although these prior art safety features may indeed fulfill their intended purposes, they all include their own drawbacks.

The '338 patent, for instance, is relatively complex and requires injection devices having openings situated at different heights. The '465 and '932 patents, on the other hand, both include structure that can lead to damage of an injection device tip if the injection device is not held properly during attempted insertion into the pump. Specifically, the '932 patent includes only a single opening, and although such reference describes the conical depression as having a smooth surface and an angle such that injection devices are guided into the single opening, such design often leads to inadvertent damage of an injection device tip during insertion. Moreover, the '465 patent teaches a screen member having a plurality of circular openings that are situated such that a plurality of “flats” (solid surfaces) extend therebetween. During insertion, if the injection device tip hits the relatively large solid surface between the circular openings, there is the potential of damage to the injection device tip.

Therefore, there exists a need for an improved safety feature for preventing inadvertent bolus dosages.

### BRIEF SUMMARY OF THE INVENTION

A first aspect of the present invention is an implantable pump. According to one embodiment, the pump includes a housing having a replenishment port and a bolus port, a cover disposed on the housing, the cover partially covering the bolus port and including an entry opening allowing for entry into the bolus port, and a screen member associated with the entry opening, the screen member including a plurality of square holes sized so as to prevent entry of an injection device having a larger size into the bolus port.

According to other embodiments of the first aspect, the implantable pump may include additional and/or modified components. For instance, the replenishment and bolus ports may be covered by septa. The bolus port may extend around the perimeter of the housing, and the housing may be circular with the entry opening being an arcuate portion. Still further, the entry opening may include sloped walls and bounding walls disposed at its ends. The bounding walls may converge towards the replenishment opening. The square holes of the screen member may include sloped walls. The screen member may be constructed of a metallic material, may be a wire mesh, may be disposed between the housing and the cover, may be press fit within the entry opening, and may include a flange. Additionally, the cover may further include a replenishment opening allowing for entry into the replenishment port, and the injection device may be a needle or syringe.

A second aspect of the present invention is a screen member for use in an implantable pump. According to one embodiment, the screen member includes a body having a plurality of square holes formed therethrough and a flange, wherein the square holes include sloped walls defining a minimum size so as to prevent entry of an injection device having a larger size therein. The body may be constructed of a metallic material, may be a wire mesh, may include a flange, and may be of an arcuate shape.

A third aspect of the present invention is another implantable pump. The pump includes a housing having a replenishment port and a bolus port and a plurality of pins partially covering the bolus port, the plurality of pins defining a plurality of holes sized so as to prevent entry of an injection device having a larger size into the bolus port. The pump may further include a cover covering the housing, and the cover may define an entry opening allowing access to the bolus port. The housing may be circular and the entry opening may be an arcuate portion. The entry opening may include sloped walls and bounding walls disposed at its ends. The plurality of pins may be disposed within hole formed in the sloped walls and bounding walls. The plurality of holes may be defined by the plurality of pins are square shaped. The pins may be constructed of a metallic material. The injection device may be a needle or syringe.

### BRIEF DESCRIPTION OF THE DRAWINGS

A more complete appreciation of the subject matter of the present invention and the various advantages thereof, can be realized by reference to the following detailed description in which reference is made to the accompanying drawings in which:

FIG. 1 is a perspective view of an implantable pump in accordance with the present invention.

FIG. 2 is a perspective view of a constant flow portion of the implantable pump shown in FIG. 1.

FIG. 3 is a top view of a control module portion of the implantable pump shown in FIG. 1.

FIG. 4 is another perspective view of the control module portion shown in FIG. 3, with specific focus on a bolus port.

FIG. 5 is another perspective view similar to that of FIG. 4, with a screen member removed therefrom.

FIG. 6 is a perspective view of a screen member of the implantable pump shown in FIG. 1.

FIG. 7 is a top view of the screen member shown in FIG. 6.

FIG. 8 is a perspective view of an implantable pump in accordance with another embodiment of the present invention.

FIG. 9 is a top view of the implantable pump shown in FIG. 8.

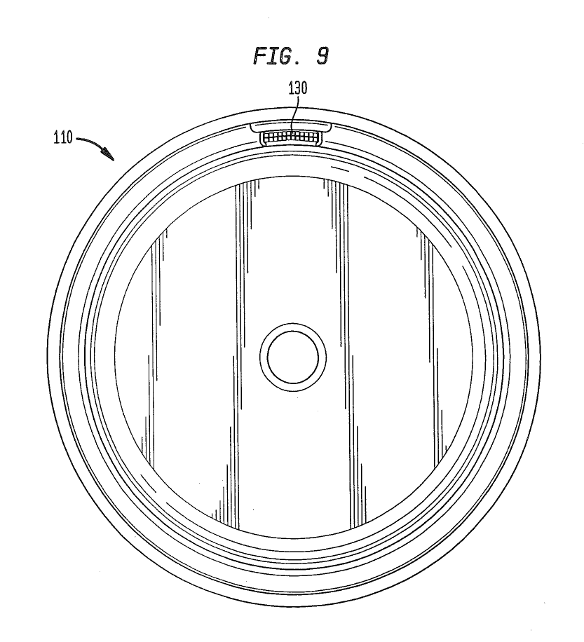

FIG. 10 is a cross-sectional view highlighting a screen member and septum of the implantable pump shown in FIG. 8.

FIG. 11 is a perspective view of the screen member partially shown in FIG. 10.

FIG. 12 is a perspective view of a cover for an implantable pump in accordance with another embodiment of the present invention.

FIG. 13 is an enlarged view of an opening in the cover shown in FIG. 12 and associated screen member.

FIG. 14 is a cross-sectional view highlighting the opening and screen member shown in FIG. 13.

FIG. 15 is a top view of the screen member included in the opening shown in FIGS. 12-14.

FIG. 16 is a perspective view of an implantable pump in accordance with another embodiment of the present invention.

FIG. 17 is a top enlarged view of an opening of the pump shown in FIG. 16.

FIG. 18 is an enlarged perspective view of a top portion of the pump shown in FIG. 16, highlighting the opening shown in FIG. 17.

FIG. 19 is an exploded perspective view of an opening included in an implantable pump in accordance with another embodiment of the present invention.

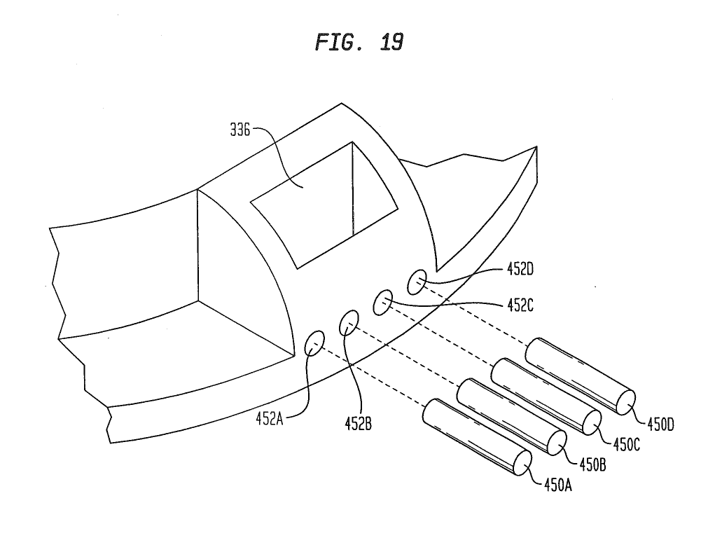

FIG. 20 is a top view of the opening shown in FIG. 19.

### DETAILED DESCRIPTION

In describing the preferred embodiments of the subject matter illustrated and to be described with respect to the drawings, specific terminology will be used for the sake of clarity. However, the invention is not intended to be limited to any specific terms used herein, and it is to be understood that each specific term used herein includes all technical equivalents which operate in a similar manner to accomplish a similar purpose.

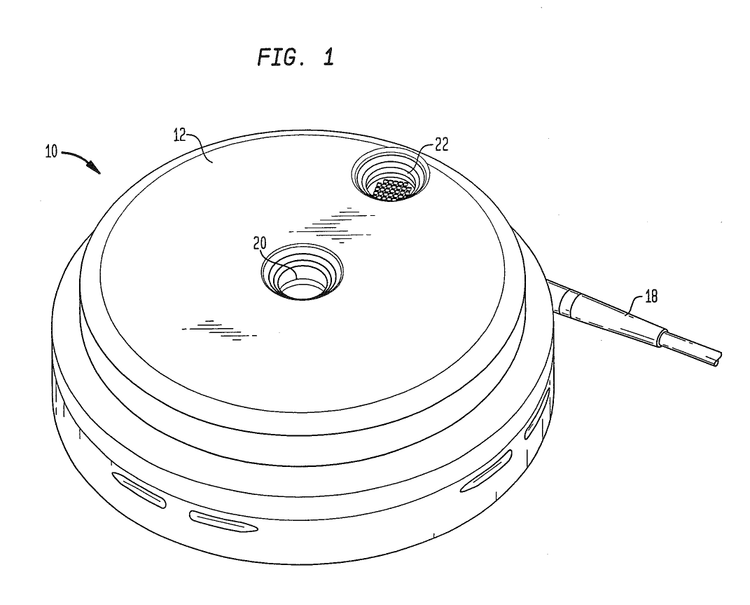

Referring to the drawings wherein like reference numerals refer to like elements, there is shown in FIG. 1, in accordance with an embodiment of the present invention, an implantable pump designated generally by reference numeral 10. Pump 10 includes, inter alia, a cover 12 and a constant flow portion 14, a control module 16 and a catheter 18. As best shown in FIG. 1, cover 12 includes a replenishment opening 20 through which the pump can be refilled with an active substance and a catheter direct access opening 22 through which a direct injection to the patient can be performed. This operation will be discussed further below.

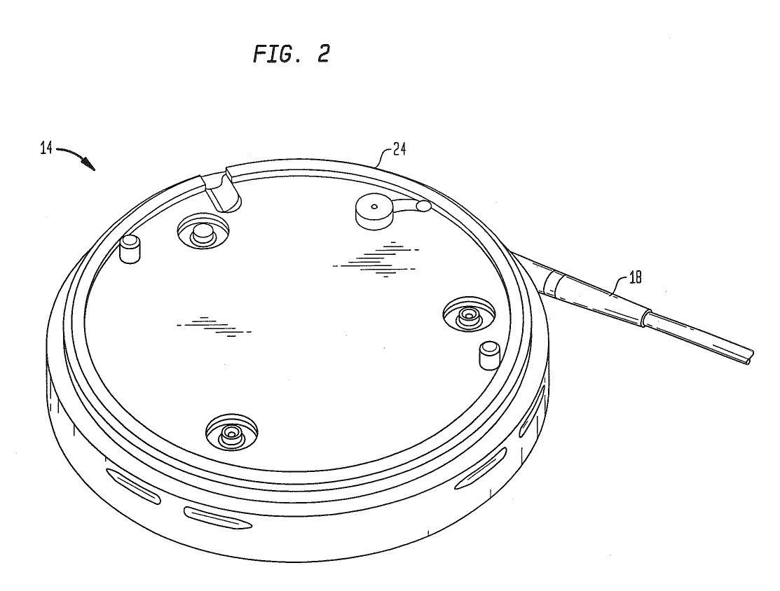

As is shown in FIG. 2, constant flow portion 14 includes, inter alia, a bolus opening or catheter direct access port (not labeled) over which is disposed a seal 24. This opening allows for an active substance to be directly injected into catheter 18. In addition, constant flow portion 14 includes other openings which cooperate with control module 16. For the purposes of this application, the components for providing a bolus dose or direct injection will be focused upon.

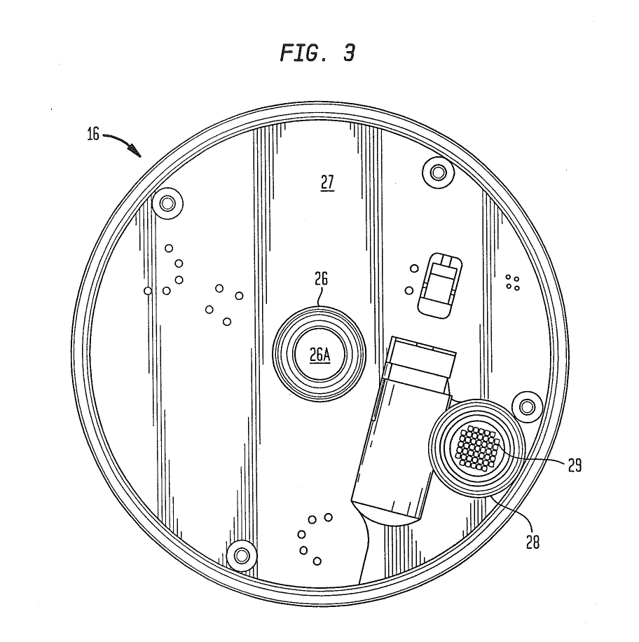

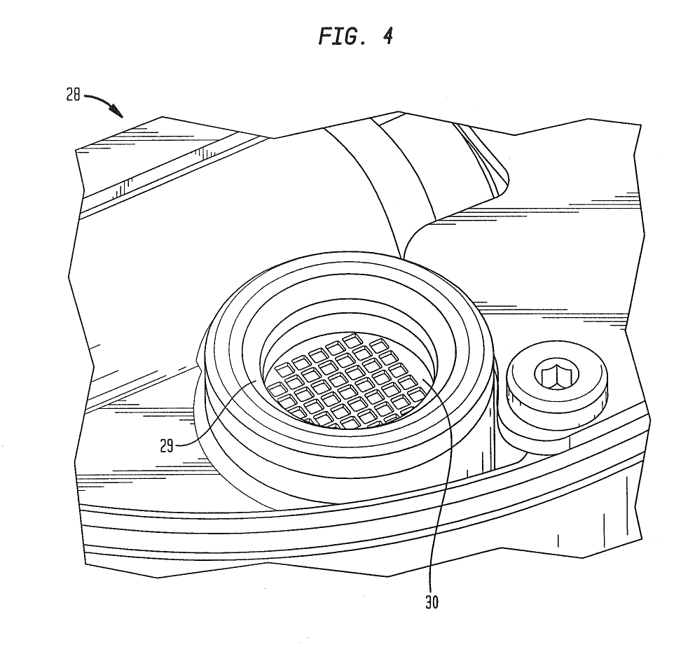

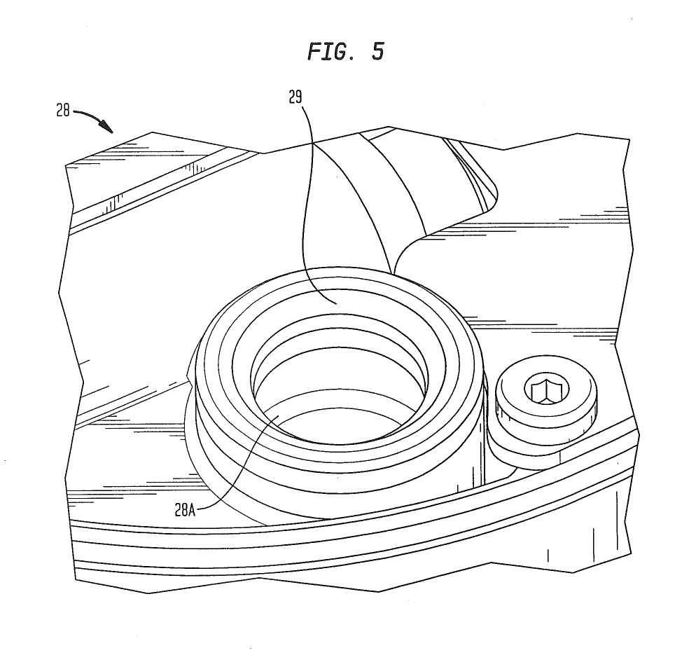

FIG. 3 is a top view of control module 16, in which refill structure 26 and bolus structure 28, among other elements, can be seen. Structure 26 houses at its center a refill septum 26a, while structure 28 houses at its center a bolus septum 28a (the latter best shown in FIG. 5). Both structures are upstanding circular walls having rounded, angled or sloped inner surfaces. Specifically, refill structure 26 includes a sloped inner wall 27 and bolus structure 28 includes a sloped inner wall 29, which both preferably aid in guiding an injection device towards the center of the structure. Inner wall 29 is better shown in FIGS. 4 and 5, with inner wall 27 employing a similar configuration. Although both structures 26 and 28 are shown as nearly identical structures, such need not be the case. Likewise, although both shown as circular in shape, structures 26 and 28 may be of any shape suitable for inclusion in pump 10.

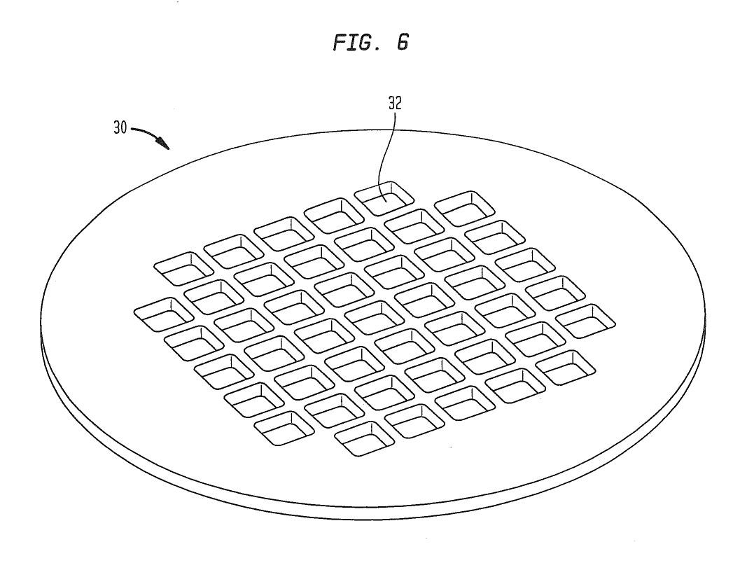

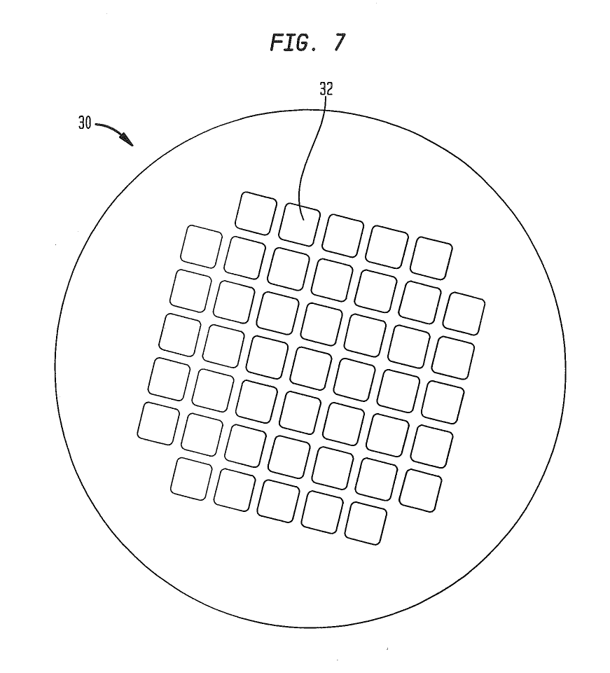

As shown in FIG. 4, septum 28a within structure 28 is overlaid with a screen member 30. FIG. 5 depicts a similar view, with screen member 30 removed, thereby depicting septum 28a and its cooperation with structure 28. FIGS. 6 and 7 depict screen member 30 alone. As shown in those figures, screen member 30 is disc-shaped and includes a plurality of openings 32. Each opening 32 exhibits a square shape and is preferably sized to only allow a certain sized injection devices therethrough. The square shape minimizes the size of the “flats” between each opening to thereby minimize the potential for tip damage during injection device insertion. The remainder of screen member 30 is preferably solid.

Screen member 30 may be constructed of many different types of materials, including metallic materials and polymeric materials. However, the member is preferably designed so as to be hard enough to prevent an injection device from penetrating through any of its solid portions. Although shown as disc-shaped (i.e., circular and flat), screen member 30 can employ many different shapes, including, but not limited to, flat structures that employ a square, rectangle, elongate curved, or oval shape, as well as non-flat structures of similar shapes. Moreover, it is to be understood that the particular opening 32 layout of the screen member shown in FIGS. 6 and 7 is but one configuration. Other embodiment screen members may include more or less openings, and it is contemplated to provide an opening array that substantially fills the screen member. Openings 32 may themselves vary in shape and size. For instance, openings 32, like certain embodiments discussed below, may employ angled or sloped walls (extending from an upper surface of the screen member to a lower surface thereof) that aid in insertion of an injection device.

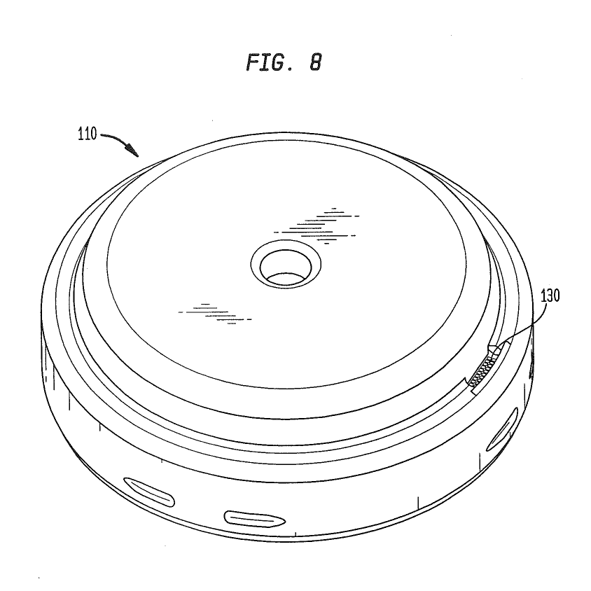

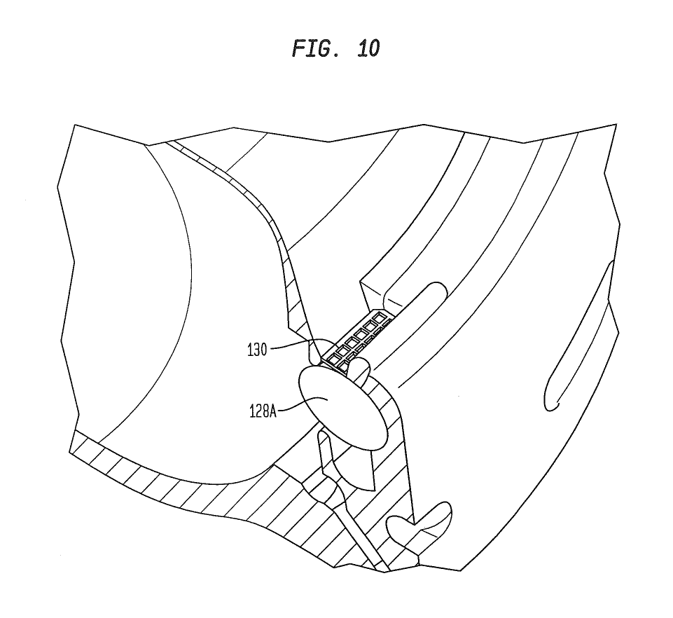

FIG. 8 depicts another embodiment implantable pump designated by reference numeral 110. Pump 110 is much like that of pump 10, save for the manner in which a bolus does or direct injection is applied. Specifically, an elongate and partially curved screen member 130 is provided on the circumferential edge of the pump, and covers an elongate ring septum 128a (best shown in FIG. 10. Screen member 130 is much like that of above-discussed screen member 30 except for its shape and its inclusion of apertures 134a and 134b. The shape is such that it can cover the like-shaped ring septum 128a. Apertures 134a and 134b on the other hand aid in the fixation of the screen member within the remainder of pump 110. Like in connection with screen member 30, screen member 130 (including openings 132) may be of many different sizes and shapes and constructed of many different materials. It is noted that although screen member 130 is shown as only extending along a portion of the perimeter of pump 110, it can extend as much as entirely therearound. However, keeping the access area relatively short allows for the majority of septum 128a to be covered, which acts so as to focus on a single area of the pump for use in providing a bolus dose. This adds an additional safety feature in that a doctor or other medical professional must identify the particular area in which to attempt injection.

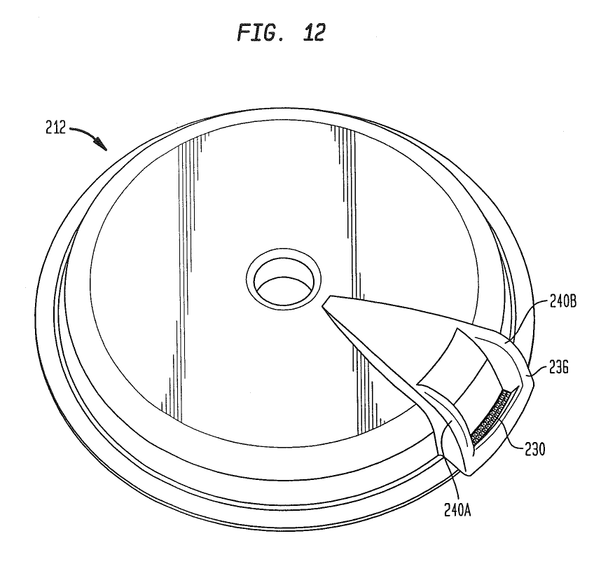

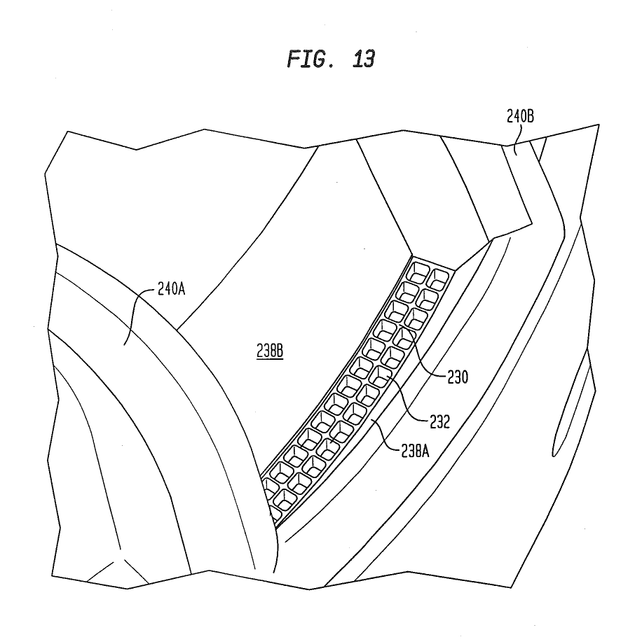

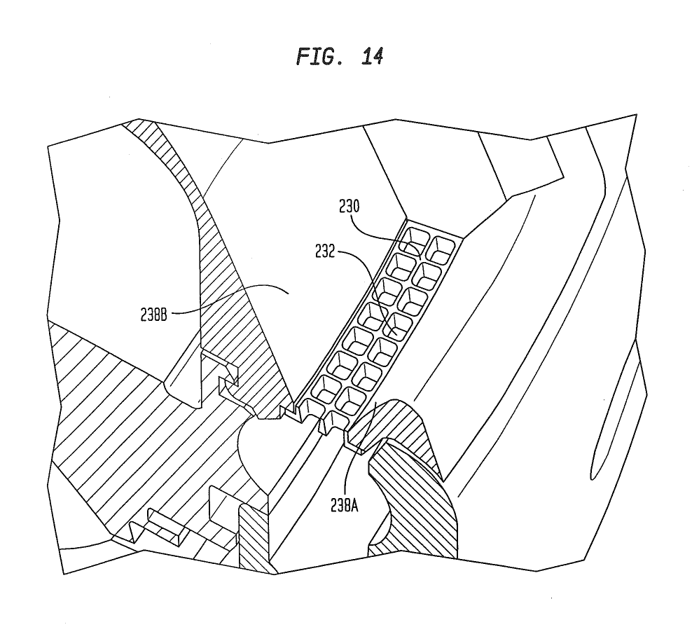

FIG. 12 depicts an alternate design similar to that of pump 110. Specifically, FIG. 12 depicts an alternate cover 212 employing an arcuate screen member 230 covering a ring septum (not shown). Screen member 230 includes a plurality of openings 232. Moreover, the screen member is disposed within an arcuate opening 236 that is defined by side walls 238a and 238b (best shown in FIGS. 13 and 14) which essentially slope towards one another so as to aid in guiding an injection device towards screen member 230. Cover 212, as shown, also includes walls 240a and 240b, which bound opening 236 on either side. These walls preferably converge near the center of pump 210, so that they provide a landmark that helps a doctor or other medical professional to identify opening 236 while pump 210 is implanted in a patient, i.e., through the skin.

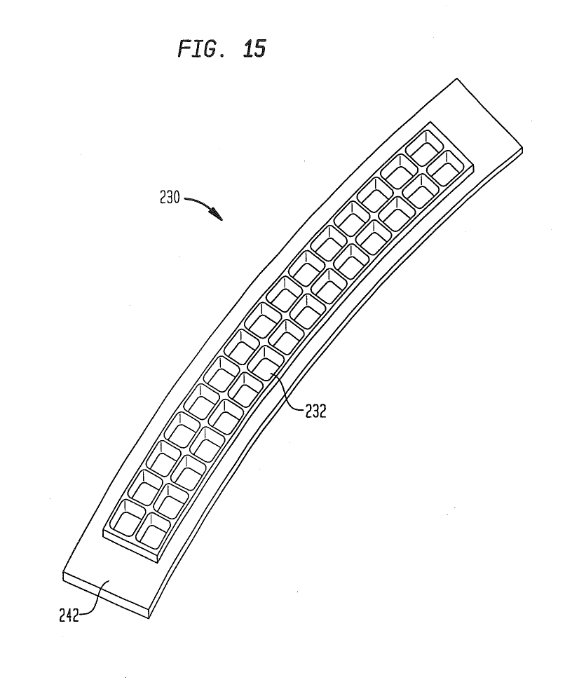

As is shown in FIG. 15, openings 232 of screen member 230 are preferably square shaped and include similarly sloped side walls. Again, this aids in the introduction of an injection device through the holes, while also providing a minimum opening size at the base of holes 230. That minimum opening size is preferably such that only injection devices having a certain diameter or smaller can be inserted therethrough and into the bolus port. Thus, similar devices having a larger diameter (e.g., those generally designated for a replenishment procedure) are prevented from extending through the openings. As is also shown in FIG. 15, screen member 230 includes a flange 242 which extends around the plurality of holes 232 and allows for the screen member to be press fit within opening 236. Screen member 230 may also be mounted in other fashions, including through the use of adhesives, welds, or the like. In a preferred embodiment, screen member 230 is constructed of metal and cooperates with cover 212 which is constructed of PEEK. However, as in the above-discussed embodiments, it is contemplated to vary the materials of either of those elements, or the remainder of pump 210. For instance, it is contemplated to form screen member 230 from PEEK or another polymer. It is also contemplated to create screen member 230 (or any of the other screen members discussed herein) as a wire mesh, which may or may not include sloping openings 232. In any event, the square shape of each of openings 232 and the rectangular shape of screen member 230 itself, minimizes the size of the “flats” between each opening to thereby minimize the potential for tip damage during injection device insertion.

During the administration of a bolus dose, a doctor or other medical professional would select a properly sized injection device and insert such through the patient's skin over the area of opening 236. As is noted above, the structure of that opening, including walls 240a and 240b, is such that it preferably can be felt from outside of the patient's skin, thereby providing a landmark for the initial insertion of the syringe. Walls 238a and 238b again are sloped so as to preferably guide the syringe towards screen 230. Once in contact with screen 230, the tip of the injection device is preferably then guided into one of openings 232, which also include sloped walls to aid in the smooth insertion of the syringe. If the injection device is properly sized, it is allowed to pass through openings 232 and ultimately through the ring septum (not shown) and into the bolus opening (also not shown). However, if the injection device includes too large a diameter, like that meant for a replenishment process, the diameter of openings 232 act as a block to the insertion of the syringe. Moreover, the configuration of openings 232 acts to soften the force that occurs when an injection device is “rejected” from insertion. In certain cases, the tip of the injection device may enter opening 232, but not pass therethrough. This prevents inadvertent damage to the tip of an injection device that may thereafter still be utilized in, for instance, a replenishment process. Although this method has been discussed in connection with the embodiment depicted in FIGS. 12-15, only minor variations of such method would equally apply to the other embodiments discussed herein.

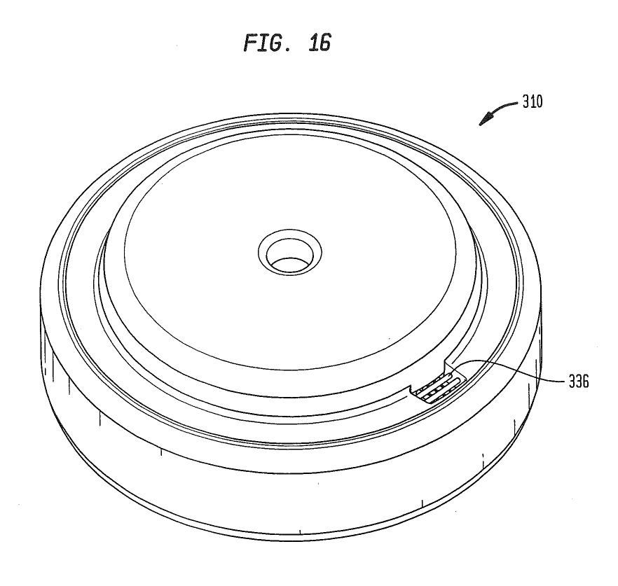

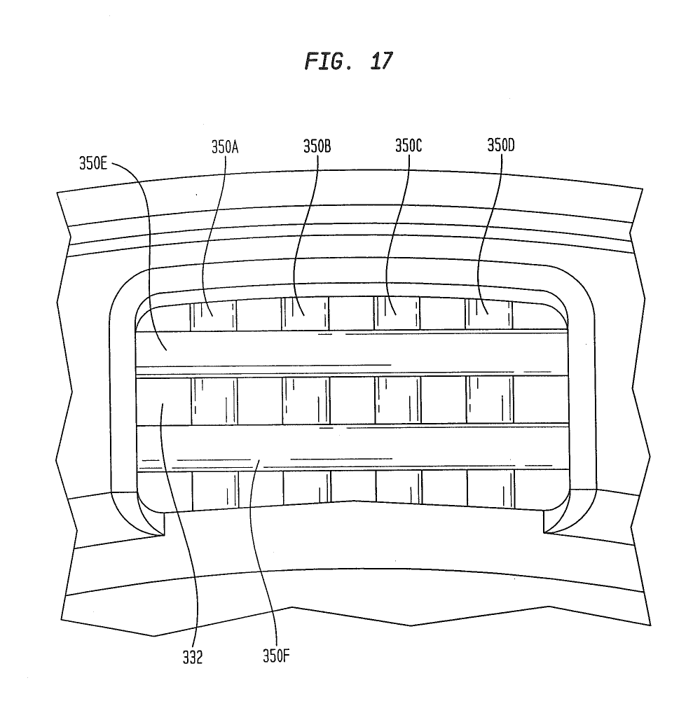

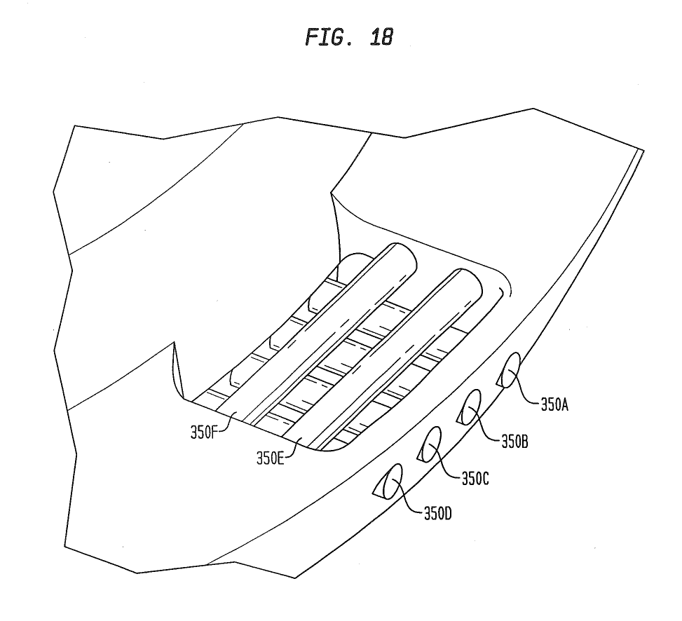

Another embodiment pump 310 is depicted in FIGS. 16-18. As in certain of the above-discussed embodiments, pump 310 includes an arcuate entry opening 336 which allows for access to a ring septum (not shown). However, instead of a screen member defining a plurality of openings for allowing/preventing an injection device from passing therethrough, opening 336 includes a plurality of pins 350a-f (best shown in FIGS. 17 and 18) defining openings 332 therebetween. Specifically, pins 350a-d extend in a first direction and pins 350e-f extend in a second direction perpendicular to the first direction. This defines a plurality of square-shaped openings 332, in a similar fashion to the above-discussed screen members.

Opening 336 again only extends along a portion of the perimeter of pump 310, but it is to be understood that such could extend as much as entirely therearound. Opening 336 preferably also includes side walls which are rounded so as to aid in guiding an injection device towards openings 332. The rounded nature of pins 350a-f also aids in the introduction of an injection device through openings 332, while also providing a minimum opening size at the base of such openings. That minimum opening size is preferably such that only injection devices having a certain diameter or smaller can be inserted therethrough and into the septum. Thus, like in the above-discussed screen member, injection devices having a larger diameter are generally designated for a replenishment procedure, while injection devices of a smaller size are utilized for administering a bolus dose as described above.

In the embodiment shown in FIGS. 16-18, a cover 312 of pump 310 includes holes for receiving pins 350a-d therein (best shown in FIG. 18). Preferably, pins 40a-d are sized so as to snugly fit within such holes. However, it is also contemplated to utilize other means of fixating the pins within the holes, including the use of adhesives, welding, screw connections, or the like. Moreover, although pins 350a-f are shown as being of approximately the same size, it is contemplated to provide some pins that are larger or smaller than others. In a preferred embodiment, pins 350a-f are constructed of metal and cooperate with a cover 312 constructed of PEEK. It is contemplated to vary the materials of any of those elements, or the rest of pump 310. For instance, it is contemplated to form pins 350a-f from PEEK or another polymer. In any event, like in the case of the above-discussed screen members, the square shape of each of holes 332 and the rectangular shape of opening 336 minimizes the size of the “flats” between each opening to thereby minimize the potential for tip damage during injection device insertion. The administration of a bolus dose is preferably carried out in a similar fashion as in the above-embodiment.

During construction of pump 310 in accordance with this second embodiment, pins 350a-f are inserted within the holes formed in cover 312. It is to be understood that certain of those holes may be blind holes, i.e., only extending partially through the wall in which they are formed. In such a case, a corresponding hole would preferably extend entirely through the opposite wall, and the pin insertion would begin at the wall with the complete hole. This results in a situation where the pin could not be inserted too far, i.e., entirely through a hole. As is noted above, pins 350a-f can be affixed within the holes of cover 312 by virtue of a tight fit therewith or other means may be employed. Additionally, in the embodiment shown in FIGS. 16-18, pins 350e-f are situated above pins 350a-d. Of course, any variation of this construction can be employed, including, pins 350a-d being situated above pins 350e-f.

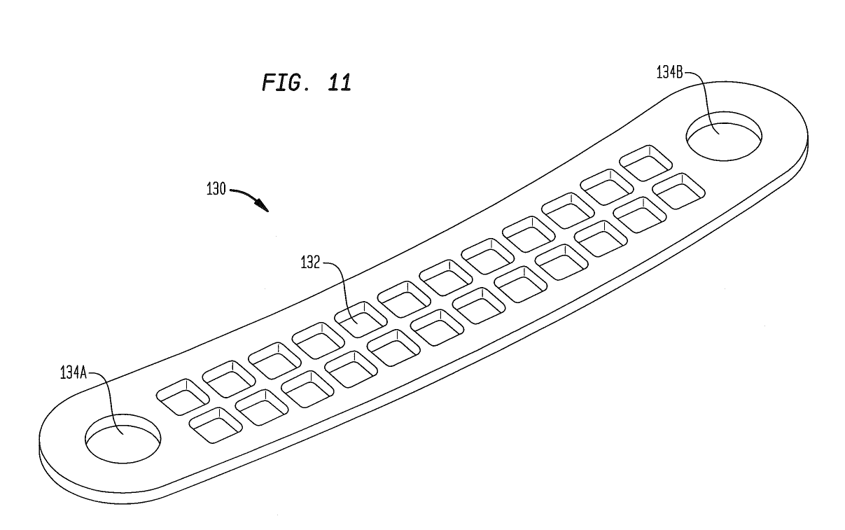

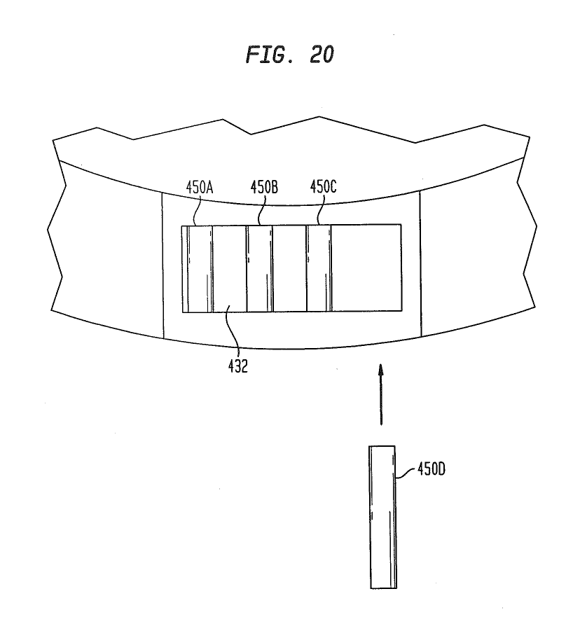

FIGS. 11 and 12 depict a variation of pin configuration employed in pump 310, where only pins extending in a single direction are employed. Specifically, as shown, only pins 450a-d are placed within holes 452a-d formed within a structure forming an opening 336. The overall size of opening 336 is such that the inclusion of just these four pins results in the creation of a plurality of square-shaped openings 432 (best shown in FIG. 20). Such a design can reduce the overall amount of materials utilized, as well as the time needed for assembly of pump 410. While square openings are discussed above in connection with each of the embodiments discussed and shown, other shaped openings may be employed. For instance, it is contemplated to utilize a screen member or configuration of pins that create rectangular openings.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An implantable pump comprising:
a housing having a replenishment port and a bolus port;
a cover disposed on the housing, the cover partially covering the bolus port and including an entry opening allowing for entry into the bolus port; and
a screen member associated with the entry opening, the screen member including a plurality of square holes sized so as to prevent entry of an injection device having a larger size into the bolus port.

2. The implantable pump of claim 1, wherein the replenishment and bolus ports are covered by septa.

3. The implantable pump of claim 2, wherein the bolus port extends around the perimeter of the housing.

4. The implantable pump of claim 3, wherein the housing is circular and the entry opening is an arcuate portion.

5. The implantable pump of claim 4, wherein the entry opening includes sloped walls.

6. The implantable pump of claim 5, wherein the entry opening includes bounding walls disposed at its ends.

7. The implantable pump of claim 6, wherein the bounding walls converge towards the replenishment opening.

8. The implantable pump of claim 1, wherein the square holes of the screen member include sloped walls.

9. The implantable pump of claim 8, wherein the screen member is constructed of a metallic material.

10. The implantable pump of claim 9, wherein the screen member is a wire mesh.

11. The implantable pump of claim 1, wherein the screen member is disposed between the housing and the cover.

12. The implantable pump of claim 11, wherein the screen member is press fit within the entry opening.

13. The implantable pump of claim 12, wherein the screen member includes a flange.

14. The implantable pump of claim 1, wherein the cover further includes a replenishment opening allowing for entry into the replenishment port.

15. The implantable pump of claim 1, wherein the injection device is a needle or syringe.

16. A screen member for use in an implantable pump comprising:
a body having a plurality of square holes formed therethrough and a flange,
wherein the square holes include sloped walls defining a minimum size so as to prevent entry of an injection device having a larger size therein.

17. The screen member of claim 16, wherein the body is constructed of a metallic material.

18. The screen member of claim 17, wherein the body is a wire mesh.

19. The screen member of claim 16, wherein the body further includes a flange.

20. The screen member of claim 16, wherein the body is of an arcuate shape.

21. An implantable pump comprising:
a housing having a replenishment port and a bolus port; and
a plurality of pins partially covering the bolus port, the plurality of pins defining a plurality of holes sized so as to prevent entry of an injection device having a larger size into the bolus port.

22. The implantable pump of claim 21, further comprising a cover covering the housing.

23. The implantable pump of claim 22, wherein the cover defines an entry opening allowing access to the bolus port.

24. The implantable pump of claim 23, wherein the housing is circular and the entry opening is an arcuate portion.

25. The implantable pump of claim 24, wherein the entry opening includes sloped walls.

26. The implantable pump of claim 25, wherein the entry opening includes bounding walls disposed at its ends.

27. The implantable pump of claim 26, wherein the plurality of pins are disposed within hole formed in the sloped walls and bounding walls.

28. The implantable pump of claim 21, wherein the plurality of holes defined by the plurality of pins are square shaped.

29. The implantable pump of claim 21, wherein the pins are constructed of a metallic material.

30. The implantable pump of claim 21, wherein the injection device is a needle or syringe.
